(19) United States
(12) Patent Application Publication
(10) Pub. No.: US 2025/0090793 A1
(43) Pub. Date: Mar. 20, 2025

(54) SYSTEM AND METHOD FOR ZENLOCITY THERAPEUTIC SPHERES RELEASE

(71) Applicant: Zhixin Wang, Tampa, FL (US)

(72) Inventor: Zhixin Wang, Tampa, FL (US)

(21) Appl. No.: 18/370,333

(22) Filed: Sep. 19, 2023

(51) Int. Cl. A61M 21/02; A61M 21/00; A63B 23/16; A63B 43/00

(52) U.S. Cl. CPC A61M 21/02; A63B 23/16; A63B 43/002; A61M 2021/0022; A61M 2205/056

(57) ABSTRACT

A system and method for therapeutic spheres with any number of protrusion designs surfaces to help train distal sensation receptors to promote sensation serve nerve recovery whereby circling the balls in one's hand and/or tracing the prominence of the ball surface can help mental focus and reduce fidget activity.

### FIELD OF DISCLOSURE

The overall invention relates generally to hand exercisers and in particular the present invention relates to a therapeutic ball for hand/digits range of motion exercises, sensory training, relieving stress, and helping with meditation.

### BACKGROUND

As individuals age, their joints, including those in the hands and fingers, naturally tend to become less flexible. As time goes by, the protective cartilage in these joints may start to wear down, leading to a condition known as osteoarthritis, which is the most prevalent form of arthritis. Although osteoarthritis often affects weight-bearing joints like the knees and hips, it can also impact the hands and fingers. The deterioration of cartilage causes the joint space to narrow, resulting in stiffness, discomfort, and restricted range of motion.

Joints are kept smooth and pain-free by synovial fluid, which may decrease in production as individuals age, resulting in decreased joint lubrication. Inadequate lubrication can contribute to stiffness and discomfort in the hands and fingers, especially as people experience a gradual loss of muscle mass and strength, a condition known as sarcopenia. Weakening hand and finger muscles can reduce joint support, leading to increased stress on the joint structures and potential stiffness.

Over a person's lifetime, the hands and fingers undergo significant repetitive movements, stress, and wear and tear. These cumulative stresses can ultimately lead to joint damage and stiffness. Activities such as gripping, twisting, or repetitive motions can accelerate the degenerative changes in the hands and fingers. Additionally, certain types of arthritis, like rheumatoid arthritis, can cause joint inflammation over time, potentially resulting in deformities such as misalignment and the formation of nodules. These deformities can contribute to joint stiffness and impaired hand function.

While aging is a significant risk factor for hand and finger stiffness and arthritis, there are also pathological conditions like stroke, Parkinson's disease, and traumatic brain injuries that can cause dysfunction in the central nervous system. Consequently, individuals with such conditions may experience stiffness, muscle atrophy due to nerve damage-induced muscle innervation, loss of sensation from peripheral nerve damage, and soft tissue contractures, all of which can be highly debilitating.

Hand and finger stiffness and reduced range of motion can also result from injuries or surgeries involving the hand, finger, or wrist. Immobilization during the post-surgery or post-injury period can lead to soft tissue adhesions, often causing muscle disuse atrophy and reduced finger coordination and motor function. Therapeutic exercises targeting the hand and fingers can aid in rehabilitating these impairments and optimizing hand and finger function.

Hand therapy exercises that focus on enhancing hand and finger range of motion, flexibility, sensation, and strength are essential not only for individuals experiencing age-related or injury-related hand and finger stiffness and impaired function but also for patients recovering from stroke or dealing with other neurological conditions. Stroke, in particular, can result in motor impairments, weakness, coordination loss, and reduced dexterity in the affected hand. Some muscles may become tight or shortened due to lack of use, leading to muscle contractures that limit joint movement and hinder effective hand use. Consistent hand therapy exercises can help retrain the brain and nervous system, facilitating connections between neurons and promoting the recovery of motor control to strengthen muscles and improve hand and finger coordination and flexibility. Sensory deficits, such as reduced tactile sensation or altered proprioception (awareness of body position), are common issues in the stroke population as well.

Hand therapy exercises involving touch, manipulation of objects, and sensory stimulation can effectively enhance sensation and sensory integration in the affected hand. These exercises are not exclusive to the aging population with pathology or joint arthritis but are also beneficial for younger athletes with high demands for optimal hand and finger function. Depending on the specific sport or activity, athletes often sustain hand and finger injuries due to the repetitive and demanding use of their hands, such as in rock climbing, weightlifting, wrestling, and tennis. Therapeutic exercises focusing on range of motion, muscle recovery, and sensation/motor integration can aid in post-sport event recovery, injury prevention, and promoting long-lasting participation in sports. Hand and finger therapeutic exercises may also serve as part of a mental training regimen to improve athletes' mental focus.

The influence of social media platforms like Facebook, Instagram, and TikTok is likely contributing to the shortened attention spans of younger generations. Fortunately, there are various exercises and practices, such as meditation, yoga, brain training games, and mindfulness practices, that can help improve attention and focus. Hand and finger therapeutic exercises incorporating tracing techniques can also contribute to enhancing mental focus and attention.

Balls have become increasingly more popular for therapeutic and meditation purposes. Soft, spongy and flexible balls held in the hands have been used to exercise the hand muscles and joints used for gripping. Use of these balls do not provide therapy beyond the hands such as the arms and shoulders and only provide one movement for the user and thus are not suitable for improving attention and focus. Other balls are simply spheres and require the user to rotate them in their hand which also limits the use for meditation. Thus exists the need for a new therapeutic exercise and meditation ball.

### SUMMARY

The present invention invokes the concept of Zenlocity which is the use of motion by the user to find therapeutic relaxation. The invention accomplishes this goal by providing three-dimensional object articles of manufacture having physical shapes and patterns at predetermined locations for use in therapy to facilitate meditation and relaxation as well as overcome grief, depression, feelings of loss, physical pain, and/or neuropathy. In one embodiment, the three dimensional object may be a ball of any suitable construction (e.g., inflatable, solid, etc.) and shape (e.g., sphere-shaped, oblong, smooth curved surfaces, flat paneled surfaces, etc.). The shapes and patterns on the ball may comprise any number of designs, including symbols such as a lemniscate or other spiritual meditation symbols. The ball may have any number of suitable color schemes, including (but not limited to) a white ball with colored symbols, a colored ball with white symbols.

### BRIEF DESCRIPTION OF DRAWINGS

The present invention will be described by way of exemplary embodiments, but not limitations, illustrated in the accompanying drawings in which like references denote similar elements, and in which:

FIG. 1 shows an illustration of a first embodiment of the zenlocity therapeutic spheres.

FIG. 2 shows an illustration of a second embodiment of the zenlocity therapeutic spheres.

FIG. 3 shows an illustration of a third embodiment of the zenlocity therapeutic spheres.

### DETAILED DESCRIPTION

In the Summary above and in this Detailed Description, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

In the following detailed description, numerous specific details are set forth in order to provide a more thorough understanding of the one or more embodiments described herein. However, it will be apparent to one of ordinary skills in the art that the invention may be practiced without these specific details. In other instances, well-known features have not been described in detail to avoid unnecessarily complicating the description.

The present disclosure recognizes the unsolved need for a therapeutic spheres designed for hand exercises, which involves grip strength strengthening, whereby the spheres are rotated in clockwise and counterclockwise around the user's hand as well, and improves their meditation and concentration with a series of symbols and designs that the user can trace physically with their hand as well as mentally with their third eye. The tracing is done for a specific desired effect but it is important to note that a person may trace the design without moving their finger which would disturb the concentration of the user. Using these motions the spheres improve hand strength, range of motion, and fine motor skills. By targeting specific muscle groups, tendons, and joints, these hand balls provide exercises that can help restore the ability to perform everyday activities such as gripping objects, writing, buttoning clothes, or using utensils.

The spheres may have a diameter around 40 mm in diameter. However, this is non-limiting and may be adjusted to accommodate user's hands of different sizes. The spheres may be made of crystal or metal, such as iron, stainless steel, or chrome-plated steel, as well as other materials such as stone, wood, or other synesthetic materials. The choice of material may be chosen based on personal preference, aesthetics, and the desired tactile sensations during meditation. In one or more embodiments a smaller ball may be positioned inside the sphere that makes a gentle, soothing sound as it moves, which can be helpful for focusing the mind and reducing stress.

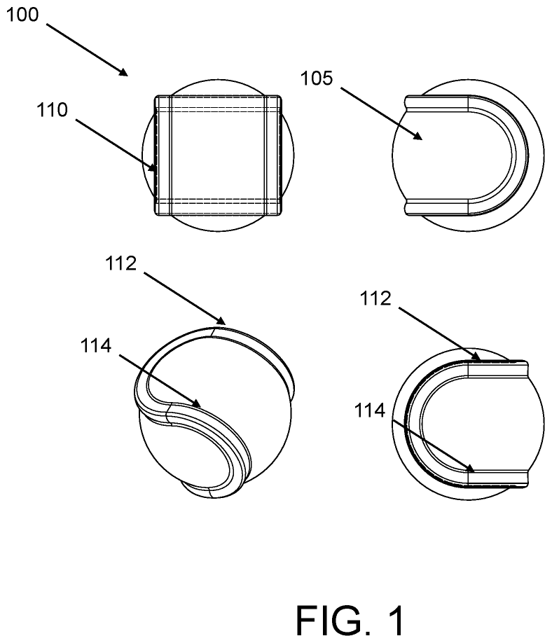

One embodiment of a therapeutic sphere in accordance with the present invention is illustrated in FIG. 1. Therapeutic sphere 100 may have a body 105 that is completely round with a smooth and continuous. Extending outward from the surface of body 105 may be a design 110. Design 110 may have two sets of protrusions 112 114 that run parallel to each other for a length horizontally along body 105. First protrusion 112 may be near the top of body 105 while second protrusion 114 may be near the bottom of body 105. The sets of protrusions 112 114 may curve inward toward each other on both ends creating a C shape and a reverse C shape when viewed from the left or right. This design was purposely created for the user to calm their mind with the shape of flowing water.

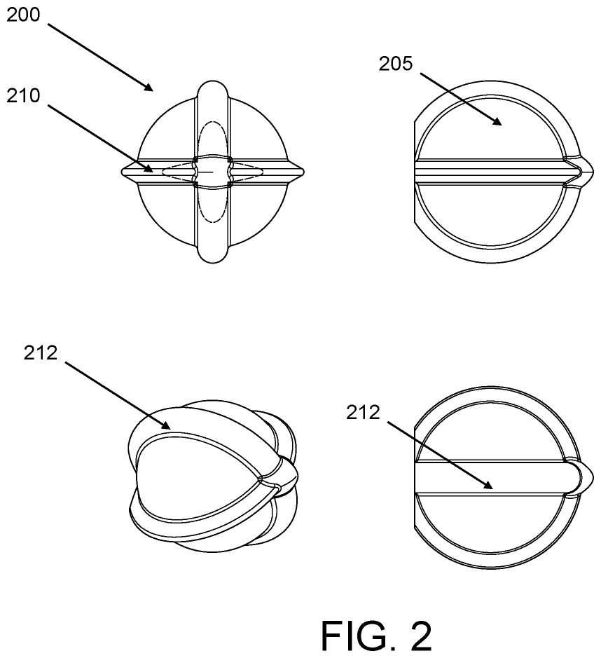

Another embodiment of a therapeutic sphere in accordance with the present invention is illustrated in FIG. 2. Therapeutic sphere 200 may have a body 205 that is completely round with a smooth and continuous. Extending outward from the surface of body 205 may be a design 210. Design 210 may have four ridges 212 that radiate outward from the center of body 205 whereby they are equally spaced apart and perpendicular to the adjacent ridge. Ridges 212 flatten and form the shape of an “X” at the base of body 205 whereby ridges 212 then extend from the base of body 205 to the top of the body 205. Ridges 212 may be slightly curved, giving it a gentle, elliptical appearance when viewed from above. It should be appreciated that four ridges is just an example for illustrations and there may be any number of ridges. This design was purposely created for focusing the mind while tracing similar to the climbing and traveling down a mountain peak.

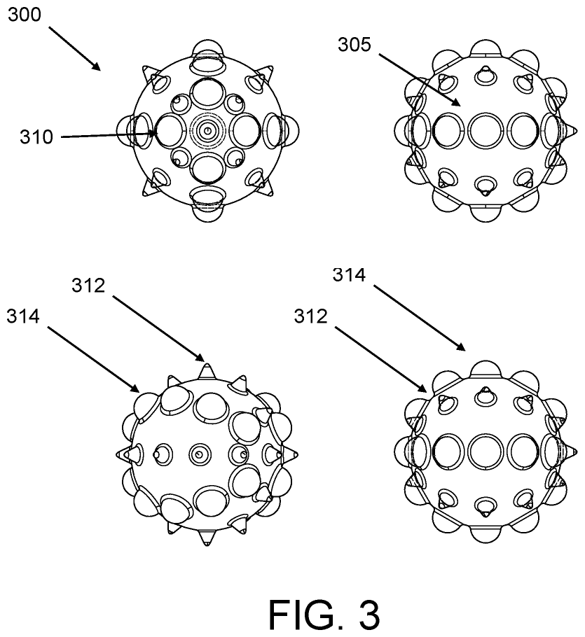

Another embodiment of a therapeutic sphere in accordance with the present invention is illustrated in FIG. 3. Therapeutic sphere 300 may have a body 305 that is completely round with a smooth and continuous. Extending outward from the surface of body 305 may be a design 310. Design 310 may be in the form of a series of cone shaped protrusions 312 that are arranged in patterns that are circles of latitude and longitude along the surface of body 305 as well as hemispherical shaped protrusions 314 that are arranged in patterns that are circles of along the surface of body at different angles or orientations from the cone shaped protrusions 312, usually but not limited to at a 45 degree diagonal angle whereby they intersect at two points on opposite sides of body 305. This design was purposely for the user to focus on conquering challenges along life that may be rocky.

During use with these therapeutic spheres in exercises can effectively stretch and elongate the muscles, preventing muscle contractures and maintaining flexibility. These hand exercises also play a crucial role in stimulating neuroplasticity, which is the brain's remarkable ability to reorganize itself and form new neural connections. By engaging in repetitive and targeted movements, stroke patients can significantly enhance neuroplasticity, facilitating the process of rewiring and relearning essential motor skills.

The act of gripping and moving these hand balls in circular motions may contribute to improved circulation and reduced swelling. Engaging in therapeutic exercises with these hand and finger balls promotes better blood flow, which in turn enhances circulation to the affected hand. This increased circulation delivers vital nutrients and oxygen to the tissues, providing valuable support for the healing process and helping to alleviate any swelling that may arise following a stroke or due to the natural aging of soft tissues.

These therapeutic spheres are designed with surfaces that specifically target the sensation nerves responsible for light touch and pinprick mechanical sensations in the hands and digits. The unique protrusions and textured surfaces on these therapeutic exercise balls are instrumental in training distal sensation receptors, thus promoting the recovery of sensory nerves. Rotating the balls within one's hand or tracing their surface's prominences can be highly beneficial for enhancing mental focus and reducing fidgeting tendencies. Beyond their physical advantages, these hand and finger therapeutic exercise balls offer notable psychological benefits. They extend beyond improving attention and promoting a sense of calm, effectively reducing anxiety and agitation. Engaging in hand therapeutic exercises with these balls can facilitate meditation for improved mental health and overall well-being. Additionally, the sense of accomplishment and boosted self-confidence that comes from observing progress in hand function can greatly motivate patients, contributing to their overall sense of well-being.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention.

The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. A method for using a therapeutic sphere, comprising steps of:
rotating the therapeutic sphere in a clockwise or counter clockwise motion; and
tracing along a design which is protruding from an exterior of the sphere that involves light touch and pinprick sensation training features.

2. The method of claim 1, wherein the tracing is done along the design with a series of hemispherical protrusions in a first series of circular arrangements and cone shaped protrusions positioned in a series of second circular arrangements at a different angle from the first series.

3. The method of claim 1, wherein the tracing is done along the design with ridges that radiate outward from the sphere wherein the ridges flatten and form a shape of an X at a base of the sphere, wherein the ridges extend from the base to a top.

4. The method of claim 2, wherein the tracing is done along the design with a first and second curved protrusions that run parallel to one another for a length and curve together at ends of the first and second curved protrusions.

5. A therapeutic sphere comprising:
a body; and
a design protruding from an outer surface of the body.

6. The therapeutic sphere of claim 5, wherein the design has hemispherical protrusions cone shaped protrusions.

7. The therapeutic sphere of claim 6, wherein the hemispherical protrusions and cone shaped protrusions are placed in arrangements that curve around the body.

8. The therapeutic sphere of claim 6, wherein the hemispherical protrusions are in a first series of circular arrangements and the cone shaped protrusions positioned in a series of second circular arrangements at a different angle from the first series.

9. The therapeutic sphere of claim 5, wherein the design has a series of ridges that radiate outward from the body.

10. The therapeutic sphere of claim 9, wherein the series of ridges form a shape of an X at a base of the sphere.

11. The therapeutic sphere of claim 9, wherein the series of ridges extend from a base of the body to a top of the body.

12. The therapeutic sphere of claim 9, wherein the series of ridges has four ridges equally spaced apart.

13. The therapeutic sphere of claim 5, wherein the design of ridges has first and second curved protrusions that run parallel to one another for a length and connect together at ends of the first and second curved protrusions wherein they curve towards one another.

14. The therapeutic sphere of claim 13, wherein the first curved protrusion is near a top of the body and the second curved protrusion is near a bottom of the body.
